(19) United States
(12) Patent Application Publication
(10) Pub. No.: US 2022/0218970 A1
(43) Pub. Date: Jul. 14, 2022

(54) METHOD FOR PROMOTING HAIR GROWTH AND IMPROVING WHITE HAIR CONDITION, AND DEVICE AND APPARATUS THEREOF

(71) Applicant: TO2M CORPORATION, Miaoli County (TW)

(72) Inventors: Garry Tsaur, Rowland Heights, CA (US); Ting-Hua Wang, Rowland Heights, CA (US)

(21) Appl. No.: 17/609,823

(22) PCT Filed: May 10, 2019
(86) PCT No.: PCT/CN2019/086385

§ 371 Date: Nov. 9, 2021

(51) Int. Cl. A61M 35/00; A61K 33/00; C01B 3/06; C01B 3/08; C01B 13/02; A45D 44/00

(52) U.S. Cl. CPC A61M 35/30; A61M 35/10; A61K 33/00; C01B 3/065; A61M 2210/06; C01B 13/0211; A45D 44/00; A61M 2202/02; A61M 2202/0208; C01B 3/08

(57) ABSTRACT

The present invention provides a method for promoting hair growth and improving white hair condition, including supplying hydrogen and oxygen to scalp and hair. The present invention further provides a hear wearable device or a hairdressing apparatus for promoting hair growth and improving white hair condition, including a gas supply device, wherein the gas supply device includes a separation layer, a hydrogen-producing composition wrapped in the separation layer, and an oxygen-producing composition wrapped in the separation layer; the hydrogen-producing composition includes: (1) a metal hydroxide and aluminum powder, or (2) a metal peroxide and aluminum powder, or (3) a hydride, or (4) a combination of the three above; and the oxygen-producing composition includes a metal peroxide.

### BACKGROUND OF THE INVENTION

### 1. Field of the Invention

The present invention relates to a method for promoting hair growth and improving white hair condition, and a device and an apparatus thereof.

### 2. Description of the Prior Art

Melanin can be divided into eumelanin and pheomelanin, which is produced by tyrosine through a series of reactions with the help of tyrosinase. Hair color is related to the melanin produced in scalp hair follicles. Asian scalp hair follicles produce eumelanin, so the hair color is black. In westerners with reddish brown hair color, hair follicles produce pheomelanin, so the hair color is reddish brown. When hair follicles are used to make melanophore of the melanin instead of making melanin, the newly produced hair will appear white, which is the so-called white hair.

In recent years, some scholars have discovered through human hair follicle cell culture and by observing human white hair that a large amount of hydrogen peroxide will be produced when the hair follicle is damaged; and when the amount of enzyme (catalase) responsible for decomposing the hydrogen peroxide in the hair follicle is insufficient to remove the hydrogen peroxide, the hair will turn white, and a large amount of hydrogen peroxide will be accumulated in the hair shaft in the white hair. What's more, the hydrogen peroxide can inhibit the activity of tyrosinase participating in the formation of melanin, thereby reducing the yield of the melanin in the hair follicles.

In addition, some studies have pointed out that scalp hypoxia will enables the hair follicles to enter a programmed cell death state, resulting in damaged and atrophic hair follicles, abnormal hair loss, less hair and finally baldness.

Modern people love beauty, and less hair and white hair are generally regarded as a symbol of aging, so when the hair is reduced or turns white, the appearance and self-confidence will be seriously affected. Therefore, when it is found that the hair is reduced or turns white, many people will actively seek ways to improve.

One of the most common way to improve white hair condition is to dye hair to mask the white hair that has grown. However, hair dyeing can only mask and improve the white hair that has already grown, and the chemical substances included in hair dye may injury human body. In recent years, some studies have pointed out that some hair dye ingredients can increase the risk of cancer.

The common way of effectively improving hair loss and reduced hair amount is hair transplantation surgery. The hair transplantation surgery is to implant hair follicles into the scalp area where the hair amount is to be increased and grow new hair after the implanted hair follicles survive. However, the hair transplantation surgery is usually expensive, and there are problems of whether the implanted hair follicles can survive successfully and maintenance.

Therefore, how to promote hair growth simply and without irritation and make the grown hair have the original hair color has become an important subject to be solved by the present invention.

### SUMMARY OF THE INVENTION

An objective of the present invention is to provide a hear wearable device for promoting hair growth, including a gas supply device, wherein the gas supply device includes a separation layer, a hydrogen-producing composition wrapped in the separation layer, and an oxygen-producing composition wrapped in the separation layer; the hydrogen-producing composition includes: (1) a metal hydroxide and aluminum powder, or (2) a metal peroxide and aluminum powder, or (3) a hydride, or (4) a combination of the three above; and the oxygen-producing composition includes a metal peroxide. The head wearable device may consist of multiple layers of materials, and an outer layer of material in the multiple layers of materials is an airtight material.

To achieve the above objective of the present invention, the separation layer may be a single layer or a composite layer; a material of the separation layer may be an air-permeable material or an airtight material; and materials of two sides of the separation layer are consistent or inconsistent.

To achieve the above objective of the present invention, the gas supply device may further include at least one conduit, and the conduit is connected to the separation layer.

To achieve the above objective of the present invention, when the hydrogen-producing composition includes a metal hydroxide and aluminum powder, the metal hydroxide in the hydrogen-producing composition includes calcium hydroxide, magnesium hydroxide, sodium hydroxide or potassium hydroxide; when the hydrogen-producing composition includes a metal peroxide or aluminum power, the metal peroxide includes calcium peroxide, magnesium peroxide, sodium peroxide or potassium peroxide; and when the hydrogen-producing composition includes a hydride, the hydride includes a hydride capable of reacting with water to produce hydrogen.

To achieve the above objective of the present invention, the metal peroxide in the oxygen-producing composition includes calcium peroxide, magnesium peroxide, sodium peroxide or potassium peroxide.

To achieve the above objective of the present invention, a better weight ratio of the hydrogen-producing composition to the oxygen-producing composition is 1:100-100:1; and a better weight ratio of the metal peroxide or the metal hydroxide to the aluminum powder is 1:100-100:1.

To achieve the above objective of the present invention, a best weight ratio of the hydrogen-producing composition to the oxygen-producing composition is 1:10-10:1; and a best weight ratio of the metal peroxide or the metal hydroxide to the aluminum powder is 1:10-10:1.

To achieve the above objective of the present invention, the hydrogen-producing composition further includes a solid acid; and the solid acid is selected from a group consisting of solid citric acid, solid lactic acid, solid oxalic acid, solid hydrochloric acid, solid phytic acid and solid silicic acid.

To achieve the above objective of the present invention, the head wearable device includes a hood, a safety helmet, a cap, a shower cap, a swimming cap or an ear temples wrapping type earmuff.

Another objective of the present invention is to provide a hairdressing apparatus for promoting hair growth, including the gas supply device as described above.

To achieve the above objective of the present invention, the hairdressing apparatus includes a hair dryer, a hair drying machine or a perming machine.

Another objective of the present invention is to provide a method for promoting hair growth, including: supplying hydrogen and oxygen to scalp and hair, wherein the promoting hair growth refers to promoting activation of hair follicles so as to grow new hair from the hair follicles.

To achieve the above objective of the present invention, hair grown by the method has the original hair color.

To achieve the above objective of the present invention, the method may be further used for improving white hair.

To achieve the above objective of the present invention, the method includes: enabling a demander to wear the head wearable device as described above or use the hairdressing apparatus as described above.

The method for promoting hair growth provided by the present invention is non-irritating, does not cause allergic reaction, and can be further used for improving white hair, thereby enabling hair follicles to produce melanin and completely eliminating the cause of white hair.

The present invention further discloses a hear wearable device or a hairdressing apparatus for promoting hair growth. The hair wearable device can keep scalp or hair in the hydrogen and oxygen environment and close to skin, so that the hydrogen and the oxygen can be easily absorbed by hair follicles, and the hair wearable device has the advantage of portability.

The hairdressing apparatus provided by the present invention can save the time of a user, so that the hair and scalp of the user are in the hydrogen and oxygen environment during hairdressing, hairdressing is preformed at the same time, and the problems of hair amount and white hair are improved.

### BRIEF DESCRIPTION OF THE DRAWINGS

FIG. 1 is a schematic diagram of a hood including a gas supply device.

FIG. 2 is a schematic diagram of the gas supply device.

FIG. 3 is an exploded view of the gas supply device.

FIG. 4 is a sectional view of the gas supply device.

FIG. 5 is a schematic diagram of installing a conduit to the gas supply device.

FIG. 6 is a 30-day test result of improving white hair at the top of the head and promoting hair growth.

FIG. 7 is a schematic diagram that the gas supply device is arranged on an ear temples wrapping type earmuff.

FIG. 8 is a 12-day test result of improving white hair of ear temples and promoting hair growth.

FIG. 9 is a schematic diagram that the gas supply device is arranged on the hairdressing apparatus.

Description of reference numerals in the accompanying drawing: 1—gas supply device; 11—separation layer; 12—hydrogen-producing composition; 13—oxygen-producing composition; 111—air-permeable structure; 14—conduit.

### DETAILED DESCRIPTION OF THE PREFERRED EMBODIMENT

The term “promoting hair growth” in this specification refers to promoting activation of hair follicles to grow new hair from the hair follicles.

The term “the original hair color” in this specification refers to the color of the air grown from the healthy, undamaged or unaged hair follicles of adult individuals after maturation; and the color of the hair may be different due to the difference in the pigment producing capability of the healthy hair follicles of each individual.

The term “white hair” in this specification refers to light-colored hair separated from the original hair color, and the light-colored hair includes gray hair, white hair or white-gray hair.

The present invention provides a method for promoting hair growth, including: continuously supplying oxygen and hydrogen to scalp and hair. The oxygen can improve the scalp hypoxia situation, and the hydrogen may promote the reaction of the following reaction formula 1 to eliminate hydrogen peroxide in the hair and hair follicles, so that the effect of promoting hair growth is achieved, and the grown hair has the original hair color. The method provided by the present invention may be further used for improving white hair condition.

H₂+H₂O₂→2H₂O Reaction formula 1:

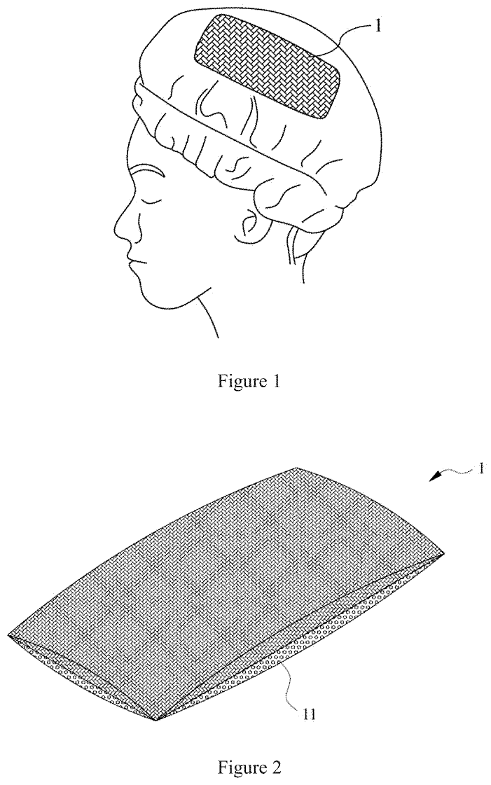

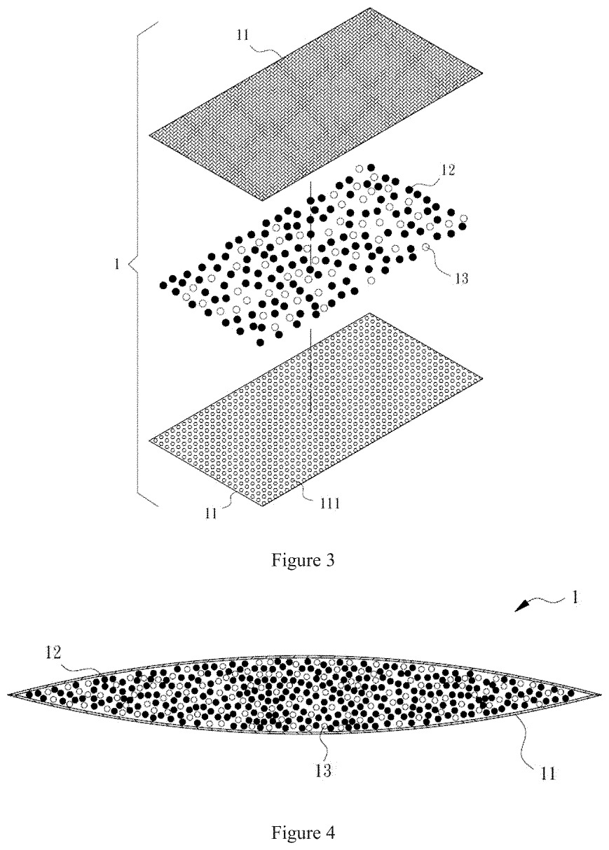

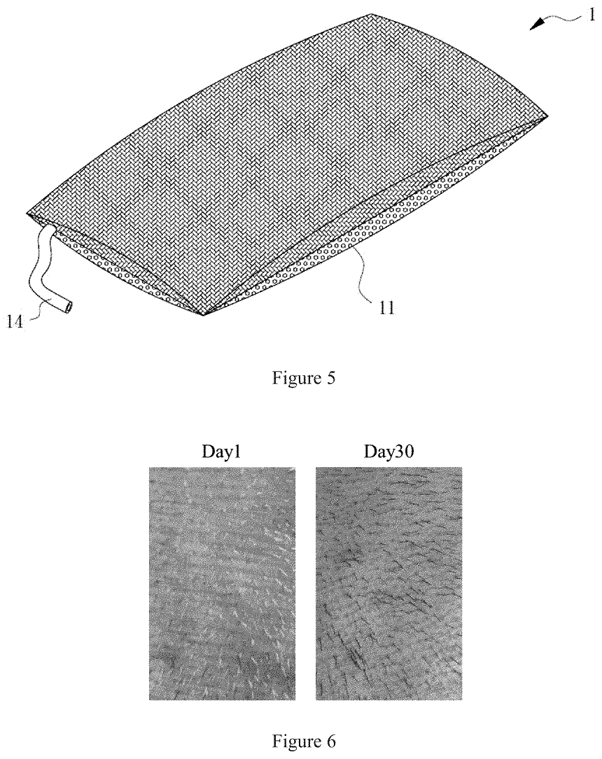

As shown in FIG. 1 to FIG. 5 and FIG. 9, the present invention further provides a head wearable device or a hairdressing apparatus for performing the method for promoting hair growth. The head wearable device or the hairdressing apparatus includes a gas supply device 1, wherein the gas supply device 1 includes a separation layer 11, a hydrogen-producing composition 12 wrapped in the separation layer 11, and an oxygen-producing composition 13 wrapped in the separation layer 11. The head wearable device may consist of multiple layers of materials, and an outer layer of material in the multiple layers of materials is an airtight material.

The separation layer 11 may be a single layer or a composite layer; a material of the separation layer 11 may be an air-permeable material or an airtight material; and materials of two sides of the separation layer 11 may be inconsistent. When the material of the separation layer 11 is an air-permeable material, the separation layer 11 is of an air-permeable structure 111 and may be used to output hydrogen and oxygen out of the separation layer 11. The gas supply device may further include at least one conduit 14, wherein the conduit 14 is connected to the separation layer 11 and may be used to guide the hydrogen and oxygen out of the separation layer 11.

The hydrogen-producing composition 12 wrapped in the separation layer 11 includes: (1) a metal hydroxide and aluminum powder; or (2) a metal peroxide and aluminum powder; (3) hydride; or (4) a combination of the three above.

The oxygen-producing composition 13 wrapped in the separation layer 11 includes a metal peroxide.

When the hydrogen-producing composition 12 includes a metal hydroxide and aluminum powder, the metal hydroxide in the hydrogen-producing composition 12 includes calcium hydroxide, magnesium hydroxide or potassium hydroxide; when the hydrogen-producing composition includes a metal peroxide or aluminum power, the metal peroxide includes calcium peroxide, magnesium peroxide, sodium peroxide or potassium peroxide; and when the hydrogen-producing composition includes a hydride, the hydride includes a hydride capable of reacting with water to produce hydrogen, such as magnesium hydride, calcium hydride and silicon hydride.

The metal peroxide in the oxygen-producing composition 13 includes calcium peroxide, magnesium peroxide, sodium peroxide or potassium peroxide.

The hydrogen-producing composition 12 and the oxygen-producing composition 13 may be granular, ingot-shaped or powdered.

When the hydrogen-producing composition 12 includes a metal hydroxide and aluminum powder, the metal hydroxide may react with the aluminum powder to generate hydrogen, as shown in the following reaction formula 2 or the reaction formula 3.

2Al+2H₂O+X(OH)₂→X(AlO₂)₂+3H₂ (X is calcium and magnesium) Reaction formula 2:

2Al+2H₂O+2Y(OH)→2YAlO₂+3H₂ (Y is sodium and potassium) Reaction formula 3:

When the hydrogen-producing composition 12 includes a metal peroxide and aluminum powder, the metal peroxide may react with water vapor in the environment to generate a metal hydroxide, as shown in the following reaction formula 4 or the reaction formula 5, and the generated metal hydroxide produces hydrogen through the reaction of the above reaction formula 2 or the reaction formula 3.

2XO₂+2H₂O→2X(OH)₂+O₂ (X is calcium and magnesium) Reaction formula 4:

2Y₂O₂+2H₂O→4Y(OH)+O₂ (Y is sodium and potassium) Reaction formula 5:

The oxygen-producing composition 13 reacts with the water vapor in the environment through the above reaction formula 4 or 5 to produce oxygen.

In order to find a better reactant ratio, 0.01-100 g of the above reactants were taken to test their gas generation efficiency, and it was found that a batter weight ratio of the hydrogen-producing composition 12 to the oxygen-producing composition 13 is 1:100-100:1 and a best weight ratio is 1:10-10:1, and a better weight ratio of the metal peroxide or metal hydroxide in the hydrogen-producing composition 12 to the aluminum powder is 1:100-100:1 and a best weight ratio is 1:10-10:1.

In addition, in order to make the hydrogen-producing composition 12 to rapidly produce a large amount of hydrogen, peroxide or hydroxide of alkali metal (sodium or potassium) with high activity may be selected as the reactant.

To reduce skin irritation and improve gas absorption, the hydrogen-producing composition 12 may further include a solid acid, and the above reaction is neutralized to make the pH value between 4 and 9, wherein the solid acid includes solid citric acid, solid lactic acid, solid oxalic acid, solid hydrochloric acid, solid phytic acid and solid silicic acid.

The gas supply device 1 provided by the present invention may be placed in the head wearable or hairdressing apparatus, the wearable device or hairdressing apparatus is full of hydrogen and oxygen through the air-permeable structure 111 or the conduit 14, and the hydrogen and the oxygen are continuously supplied to the hair and scalp, so that hydrogen peroxide in the hair and hair follicles is eliminated, and the hair loss situation caused by scalp hypoxia is avoided. The gas supply device 1 provided by the present invention may be placed outside the head wearable device or hairdressing apparatus, and the conduit 14 guides gas into the head wearable device or hairdressing apparatus, wherein the head wearable device includes a hood, a safety helmet, a cap, a shower cap, a swimming cap or an ear temples wrapping type earmuff; and the hairdressing apparatus includes a hair dryer, a hair drying machine or a penning machine.

### Embodiment I: A Hood Including a Gas Supply Device 1

As shown in FIG. 1, 1 g of sodium peroxide, 1 g of sodium hydroxide, 10 g of aluminum powder and a proper amount of oxalic acid were taken to make the gas supply device 1, and the gas supply device 1 was placed in the hood to test the effects of promoting hair growth and improving white hair condition.

Several subjects with the problems of less hair amount and white hair were recruited, the subjects were asked to wear the hood for 30 minutes to 4 hours a day, the feelings of the subjects were interviewed, and the scalp condition, the white hair and the hair growth situation of the subjects were observed and recorded.

The observation results found that the subjects did not feel uncomfortable when wearing the hood, and no redness or swelling occurred in the scalp after the hood was taken off, indicating that the method and device provided by the present invention will not irritate the scalp. The test results are shown in Table 1 and FIG. 6. After 30 days, the hair amount of the subjects were significantly increased, the white hair problem was obviously improved, and the newly grown hair had the original hair color.

TABLE 1
30-day test result of improving white hair at
the top of the head and promoting hair growth

| | | | | | | Proportion of |
|---|---|---|---|---|---|---|
| | | | | | | the amount of |
| | | | Average | | | the original- |
| | | Average | amount of | | Average | color hair in |
| | | amount of | original- | | total hair | the total hair |
| | | white hair | color hair | | amount | amount |
| 1st | day | 149 hairs | 17 | hairs | 166 hairs | 10% |
| 30th | day | 231 hairs | 5 | hairs | 236 hairs | 97% |

### Embodiment II: An Ear Temples Wrapping Type Earmuff Including a Gas Supply Device 1

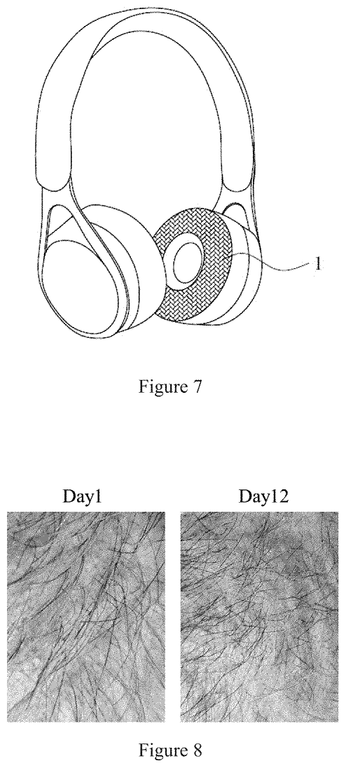

As shown in FIG. 7, 10 g of calcium peroxide, 10 g of calcium hydroxide, 1 g of aluminum powder and a proper amount of citric acid were taken to make the gas supply device 1, the gas supply device 1 was placed in the ear temples wrapping type earmuff to test the effects of promoting hair growth and improving white hair condition.

Several subjects with the problems of less hair amount and white hair were recruited, the subjects were asked to wear the ear temples wrapping type earmuff for 30 minutes to 2 hours a day, the feelings of the subjects were interviewed, and the scalp condition, the growth situation of the hair of the ear temples and the white hair condition of the subjects were observed and recorded.

The observation results found that the subjects did not feel uncomfortable at the ear temples when wearing the earmuff, and no redness or swelling occurred in skin after the earmuff was taken off. The test results are shown in Table 2 and FIG. 8. After 12 days, the white hair of the ear temples of the subjects was obviously improved, and many original-color hairs were newly grown.

TABLE 2
12-day test result of improving white hair
of ear temples and promoting hair growth

| | | | | | Proportion of |
|---|---|---|---|---|---|
| | | | | | the amount of |
| | | | Average | | the original- |
| | | Average | amount of | Average | color hair in |
| | | amount of | original- | total hair | the total hair |
| | | white hair | color hair | amount | amount |
| 1st | day | 63 hairs | 52 hairs | 115 hairs | 45% |
| 12th | day | 52 hairs | 95 hairs | 147 hairs | 64% |

### Embodiment III: Hairdressing Apparatus Including a Gas Supply Device 1

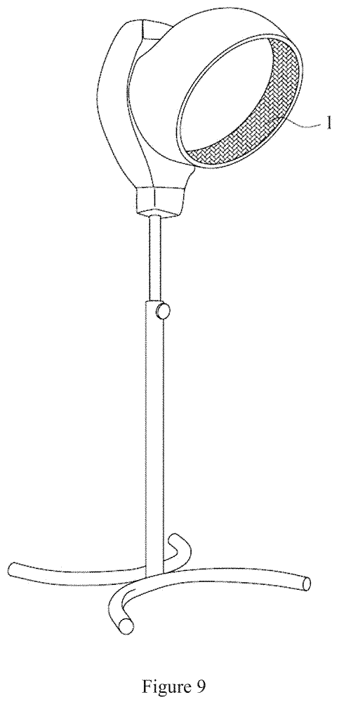

The gas supply device 1 provided by the present invention can be used with the hairdressing apparatus. As shown in FIG. 9, the gas supply device 1 is placed on the hairdressing apparatus. During hairdressing, hydrogen and oxygen are supplied to scalp and hair to make the scalp and hair breathe, and hydrogen peroxide in the hair follicles and hair is eliminated through the reaction of the reaction formula 1, so that the effects of promoting hair growth and improving white hair condition are achieved.

To sum up, the present invention provides a brand-new method for promoting hair growth, so that the hair follicles can be promoted to grow hair, the ability of the hair follicles to produce melanin can be restored, and the original-color hair can grow. Compared with the traditional method, the method provided by the present invention has the advantages of low cost, simplicity and convenience, and easy maintenance.

The method provided by the present invention may be further used to improve the problem of white hair, and has the advantages of no irritation and no damage to human body compared with the traditional method for improving the white hair condition.

In addition, the method for promoting hair growth provided by the present invention may be used together with the head wearable device or the hairdressing apparatus. The head wearable device provided by the present invention has the advantage of portability and may enable a user to promote hair growth or improve the white hair condition at any time; and the hairdressing apparatus provided by the present invention may enable a user to promote hair growth or improve the white hair condition during hairdressing, and has the advantage of saving time. The above detailed description is a specific description of the feasible embodiments of the present invention, but this embodiment is not intended to limit the patent scope of the present invention. Any equivalent implementation or modification made without departing from the technical spirit of the present invention, such as the head wearable device capable of generating hydrogen and oxygen, including a hood, a safety helmet, a cap, a shower cap, a swimming cap or an ear temples wrapping type earmuff, or the hairdressing device, including a hair dryer, a hair drying machine and a penning machine, should be included in the patent scope of the present invention.

What is claimed is:

1. A head wearable device for promoting hair growth, comprising a gas supply device, wherein the gas supply device comprises a separation layer, a hydrogen-producing composition wrapped in the separation layer, and an oxygen-producing composition wrapped in the separation layer;
the hydrogen-producing composition comprises:
(1) a metal hydroxide and aluminum power, or
(2) a metal peroxide and aluminum powder, or
(3) a hydride,
(4) a combination of the three above;
the oxygen-producing composition comprises a metal peroxide; and
the head wearable device consists of multiple layers of materials, and an outer layer of material in the multiple layers of materials is an airtight material.

2. The head wearable device according to claim 1, wherein the separation layer is a single layer or a composite layer; a material of the separation layer is an air-permeable material or an airtight material; and materials of two sides of the separation layer are consistent or inconsistent.

3. The head wearable device according to claim 1, wherein the gas supply device further comprises at least one conduit, and the conduit is connected to the separation layer.

4. The head wearable device according to claim 1, wherein when the hydrogen-producing composition comprises a metal hydroxide and aluminum powder, the metal hydroxide in the hydrogen-producing composition comprises calcium hydroxide, magnesium hydroxide, sodium hydroxide or potassium hydroxide; when the hydrogen-producing composition comprises a metal peroxide or aluminum power, the metal peroxide comprises calcium peroxide, magnesium peroxide, sodium peroxide or potassium peroxide; and when the hydrogen-producing composition comprises a hydride, the hydride comprises a hydride capable of reacting with water to produce hydrogen.

5. The head wearable device according to claim 1, wherein the metal peroxide in the oxygen-producing composition comprises calcium peroxide, magnesium peroxide, sodium peroxide or potassium peroxide.

6. The head wearable device according to claim 1, wherein a better weight ratio of the hydrogen-producing composition to the oxygen-producing composition is 1:100-100:1; and a better weight ratio of the metal peroxide or the metal hydroxide to the aluminum powder is 1:100-100:1.

7. The head wearable device according to claim 6, wherein a best weight ratio of the hydrogen-producing composition to the oxygen-producing composition is 1:10-10:1; and a best weight ratio of the metal peroxide or the metal hydroxide to the aluminum powder is 1:10-10:1.

8. The head wearable device according to claim 1, wherein the hydrogen-producing composition further comprises a solid acid; and the solid acid is selected from a group consisting of solid citric acid, solid lactic acid, solid oxalic acid, solid hydrochloric acid, solid phytic acid and solid silicic acid.

9. The head wearable device according to claim 1, comprising a hood, a safety helmet, a cap, a shower cap, a swimming cap or an ear temples wrapping type earmuff.

10. A hairdressing apparatus for promoting hair growth, comprising the gas supply device as defined in claim 1.

11. The hairdressing apparatus according to claim 10, comprising a hair dryer, a hair drying machine or a perming machine.

12. A method for promoting hair growth, comprising: supplying hydrogen and oxygen to scalp and hair, wherein the promoting hair growth refers to promoting activation of hair follicles so as to grow new hair from the hair follicles.

13. The method according to claim 12, wherein hair grown by the method has the original hair color.

14. The method according to claim 12, capable of being further used for improving white hair.

15. The method according to claim 12, comprising: enabling a demander to wear the head wearable device or use a hairdressing apparatus;
wherein the head wearable device comprising a gas supply device, wherein the hairdressing apparatus comprising the gas supply device; wherein the gas supply device comprises a separation layer, a hydrogen-producing composition wrapped in the separation layer, and an oxygen-producing composition wrapped in the separation layer;
the hydrogen-producing composition comprises:
(1) a metal hydroxide and aluminum power, or
(2) a metal peroxide and aluminum powder, or
(3) a hydride,
(4) a combination of the three above;
the oxygen-producing composition comprises a metal peroxide; and
the head wearable device consists of multiple layers of materials, and an outer layer of material in the multiple layers of materials is an airtight material.
